(19) United States
(12) Patent Application Publication
(10) Pub. No.: US 2007/0123462 A1
(43) Pub. Date: May 31, 2007

(54) Effects of tissue transglutaminase on beta-amyloid-induced apoptosis

(75) Inventors: Richard A. Cerione, Ithaca, NY (US); Joseph Wakshlag, Woodbury, CT (US); Marc Antonyak, Ithaca, NY (US)

Correspondence Address: NIXON PEABODY LLP - PATENT GROUP, CLINTON SQUARE, P.O. BOX 31051, ROCHESTER, NY, 14603-1051, US

(73) Assignee: Cornell Research Foundation, Inc., Ithaca, NY (US)

(21) Appl. No.: 11/600,660

(22) Filed: Nov. 16, 2006

Related U.S. Application Data
(60) Provisional application No. 60/737,456, filed on Nov. 16, 2005.

(51) Int. Cl. A61K 38/55; C12Q 1/48

(52) U.S. Cl. 514/12; 435/15

(57) ABSTRACT

The present invention relates to a method of inhibiting beta-amyloid-induced death of neuronal cells in a subject by inhibiting human tissue transglutaminase in the subject under conditions effective to inhibit beta-amyloid-induced death of neuronal cells. Also disclosed are methods for identifying candidate compounds suitable for inhibiting beta-amyloid-induced death of neuronal cells in a subject by identifying compounds which are capable of binding to human tissue transglutaminase as candidate compounds suitable for inhibiting beta-amyloid-induced death of neuronal cells in a subject. The present invention also relates to compounds suitable for inhibiting beta-amyloid-induced death of neuronal cells in a subject, as well as methods for designing such compounds.

This application claims the benefit of U.S. Provisional Patent Application Ser. No. 60/737,456, filed Nov. 16, 2005, which is hereby incorporated by reference in its entirety.

This invention arose out of research sponsored by the National Institutes of Health (Grant No. RO1 GM61762). The U.S. Government may have certain rights in this invention.

### FIELD OF THE INVENTION

The present invention relates to methods of inhibiting beta-amyloid-induced death of neuronal cells in a subject by inhibiting human tissue transglutaminase in the subject. The present invention also relates to methods for identifying candidate compounds suitable for inhibiting beta-amyloid-induced death of neuronal cells in a subject. Compounds suitable for inhibiting beta-amyloid-induced death of neuronal cells in a subject, as well as methods for designing such compounds, are also disclosed.

### BACKGROUND OF THE INVENTION

Tissue transglutaminase, also known as transglutaminase II or TG2 and referred to herein as “TGase”, is capable of both GTP-binding and hydrolytic activity, as well as an acyl-transferase (transamidation) activity (Di Venere et al., J. Biol. Chem. 275:3915-3921 (2000); Liu et al., Proc. Natl. Acad. Sci. USA 99:2743-2747 (2002); Zhang et al., J. Biol. Chem. 273:2288-2295 (1998)). The transamidation activity catalyzed by TGase is Ca²⁺-dependent and results in the cross-linking of glutamyl side chains to either ε-amino groups of lysine residues or to the primary amino groups of polyamines (Folk, Annu. Rev. Biochem. 49:517-531 (1980); Festus et al., Trends Biochem. Sci. 27:534-539 (2002)). TGase is ubiquitously expressed, typically at relatively low levels in the absence of extracellular stimuli, but often is up-regulated in response to retinoic acid (RA) under conditions of cellular differentiation, and when cells are confronted with various stress-related insults.

There have been a number of studies directed toward establishing the functional consequences of TGase expression and activation, both with regard to cellular differentiation and programmed cell death. Initially, it was proposed that TGase up-regulation and activation were underlying causes of apoptosis. In one study it was even suggested that TGase-catalyzed transamidation of the cell-cycle check-point regulator, the Retinoblastoma (Rb) protein, contributed to programmed cell death (Oliverio et al., Mol. Cell Biol. 17:6040-6048 (1997)). However, other findings have supported the idea that TGase is up-regulated in response to different cellular insults in order to ensure cell survival, particularly under conditions of RA-induced differentiation (Antonyak et al., J. Biol. Chem. 276:33582-33587 (2001)). Moreover, the ability of TGase to catalyze the transamidation of Rb has been shown to protect Rb from caspase-mediated proteolysis and to help extend cellular lifetime in the face of apoptotic challenges (Boehm et al., J. Biol. Chem. 277:20127-20130 (2002)).

Given these different and in some cases contradictory findings, the exact function exhibited by TGase, and in particular, whether it serves as a survival or apoptotic factor, may ultimately depend on the cell type and specific circumstances. As might be expected for a protein linked both to cell survival and apoptosis, there have been a number of reports implicating TGase in various pathological and disease states including cataracts, celiac disease, cancer, and neurodegenerative disorders, in particular both Huntington's and Alzheimer's diseases (Hidasi et al., Ann. Clin. Lab. Sci. 25:236-240 (1995); Hettasch et al., Lab. Invest. 75:637-645 (1996); Lesort et al., Prog. Neurobiol. 61:439-463 (2000); Zhang et al., Glia 42:194-208 (2003); Dewar et al., Int. J. Biochem. Cell Biol. 36:17-24 (2004); Karpuj et al., Amino Acids 26:373-379 (2004); Pepe et al., Amino Acids 26:431-434 (2004)). The possible connections between TGase and Alzheimer's disease have been especially widespread and include findings that show the cerebral tissue and spinal fluid from patients with this disease have elevated levels of TGase expression and transamidation activity (Johnson et al., Brain Res. 751:323-329 (1997); Nemes et al., Neurobiol. Aging 22:403-406 (2001); Bonelli et al., Neurobiol. Dis. 11:106-110 (2002)), and that TGase is a component of β-amyloid-rich senile plaques (Zhang et al., Acta Neuropathol. (Berl) 96:395-400 (1998)).

Given the implications for an involvement of TGase both in cell survival and cell death, coupled with the suggestions that it might have some role in Alzheimer's disease, it would be advantageous to find out whether TGase contributes to or blocks β-amyloid-induced neurotoxicity.

The present invention is directed to achieving these objectives.

### SUMMARY OF THE INVENTION

The present invention relates to a method of inhibiting beta-amyloid-induced death of neuronal cells in a subject. The method involves inhibiting human tissue transglutaminase in the subject under conditions effective to inhibit beta-amyloid-induced death of neuronal cells.

Another aspect of the present invention relates to a method for identifying candidate compounds suitable for inhibiting beta-amyloid-induced death of neuronal cells in a subject. The method involves identifying compounds which are capable of binding to human tissue transglutaminase as candidate compounds suitable for inhibiting beta-amyloid-induced death of neuronal cells in a subject.

The present invention also relates to a method for designing a compound suitable for inhibiting beta-amyloid-induced death of neuronal cells in a subject. The method first involves providing a three-dimensional structure of a crystallized human tissue transglutaminase. Then, a compound having a three-dimensional structure which will bind to one or more molecular surfaces of the human tissue transglutaminase is designed.

Another aspect of the present invention relates to a compound suitable for inhibiting beta-amyloid-induced death of neuronal cells in a subject. The compound has a three-dimensional structure which will bind to one or more molecular surfaces of the human tissue transglutaminase having a three dimensional crystal structure defined by the structural coordinates set forth in FIG. 7 of U.S. Patent Application Publication No. US 2004/0259176, which is hereby incorporated by reference in its entirety.

Tissue transglutaminase (TGase) has been implicated in both cell survival and apoptosis. The present invention describes the role of TGase in β-amyloid-induced neurotoxicity using retinoic acid (RA)-differentiated, neuronal SH-SY5Y cells. The neurotoxic activity of β-amyloid₁₋₄₂, the most abundant and naturally occurring form of β-amyloid, was shown to be reduced in RA-differentiated SH-SY5Y cells treated with the TGase inhibitor monodansyl cadaverine. Expression of wild-type TGase enhanced β-amyloid₁₋₄₂-induced apoptosis, whereas transamidation-defective TGase did not. These effects were specific for β-amyloid-treated cells, as TGase reversed the neurotoxic effects caused by hydrogen peroxide, a reactive oxygen intermediate that has been suggested to mediate β-amyloid-induced cell death (Tamagno et al., Free Radic Biol Med 35:45-58 (2003); Tamagno et al., Exp Neurol 180:144-155 (2003), which are hereby incorporated by reference in their entirety). Enhancement of β-amyloid₁₋₄₂-induced cell death by TGase was accompanied by marked increases in TGase activity in the membrane fractions and translocation of TGase to the cell surface. Overall, these findings suggest that the ability of TGase to exhibit pro-survival versus pro-apoptotic activity is linked to its cellular localization, with β-amyloid-induced recruitment of TGase to the cell surface accentuating neuronal toxicity and apoptosis.

Since the inhibition of TGase's transamidation activity prevents the augmentation of cell death, the enhanced cell death caused by the recruitment of TGase is dependent on its ability to catalyze the cross-linking of cellular proteins, i.e., transamidation. Thus, the development of small molecule inhibitors that block transamidation could have therapeutic value against neurodegenerative disorders such as Alzheimer's disease.

### BRIEF DESCRIPTION OF THE DRAWINGS

FIG. 1 illustrates the overall structure of a human tissue transglutaminase (TGase) dimer with bound GDP. TGase is shown in ribbon drawing with four distinct domains: the amino-terminal β-sandwich domain, the transamidation catalytic core domain (marked by the essential Cys-277 in ball-and-stick), and the first and second carboxy-terminal β-barrel domains. GDP is shown as a ball-and-stick model between the catalytic core domain and the first β-barrel domain. The picture was prepared with MOLSCRIPT (Kraulis, J. Appl. Crystallogr., 24:946-950 (1991), which is hereby incorporated by reference in its entirety) and RASTER3D (Merritt et al., Acta Crystallogr. D, 50:869-873 (1994), which is hereby incorporated by reference in its entirety).

FIG. 2 is the stereoview of an electron density map (2Fₒ-F_{c}, 1.2σ, GDP omitted, 2.8-Å resolution) of the GDP-binding pocket, showing one GDP molecule bound to each of the six TGase monomers within the asymmetric unit. An atomic model of the final structure is embedded in the electron density. Drawing prepared from MOLSCRIPT (Kraulis, J. Appl. Crystallogr., 24:946-950 (1991), which is hereby incorporated by reference in its entirety) and RASTER3D (Merritt et al., Acta Crystallogr. D, 50:869-873 (1994), which is hereby incorporated by reference in its entirety).

FIG. 3 shows comparisons between the atomic interactions of GDP with TGase (Left) and Ras (Right). Hydrogen bonds and ion pair interactions are shown in dashed lines. The GDP molecule is shown in ball-and-stick. TGase and Ras residues are shown in thin sticks. Drawing prepared with MOLSCRIPT (Kraulis, J. Appl. Crystallogr., 24:946-950 (1991), which is hereby incorporated by reference in its entirety) and RASTER3D (Merritt et al., Acta Crystallogr. D, 50:869-873 (1994), which is hereby incorporated by reference in its entirety).

FIG. 4 shows the transamidation active site of TGase. A close-up view of the juxtaposition of the catalytic triad consisting of Cys-277-His-335-Asp-358 and Tyr-516 relative to the guanine nucleotide-binding site. Cys-277, His-335, Asp-358, Tyr-516, and GDP are shown in ball-and-stick. Tyr-516 points toward Cys-277, the catalytic nucleophile, in the active site. The drawing was prepared by using MOLSCRIPT (Kraulis, J. Appl. Crystallogr., 24:946-950 (1991), which is hereby incorporated by reference in its entirety) and RASTER3D (Merritt et al., Acta Crystallogr. D, 50:869-873 (1994), which is hereby incorporated by reference in its entirety).

FIG. 5 shows comparison of the calcium-binding sites of TGase (light grey) and Factor XIIIa (dark grey). In Factor XIIIa, the loop involved in calcium binding is oriented toward the Ca²⁺-binding site, whereas in TG-GDP, the same loop is oriented toward GDP. The figure was prepared with MOLSCRIPT (Kraulis, J. Appl. Crystallogr., 24:946-950 (1991), which is hereby incorporated by reference in its entirety) and RASTER3D (Merritt et al., Acta Crystallogr. D, 50:869-873 (1994), which is hereby incorporated by reference in its entirety).

FIGS. 6A-B depict characterization of β-amyloid₁₋₄₂. FIG. 6A shows non-denaturing gel electrophoresis of 10 μM β-amyloid₁₋₄₂ at time zero and after 24 and 48 hours of incubation at room temperature. The β-amyloid was either directly reconstituted in Me₂SO or was pre-treated with hexafluoroisopropanol (HFIP) and dessicated prior to reconstitution in Me₂SO. FIG. 6B shows an immunoblot for activated caspase-3, as a function of time of incubation at 37° C. with β-amyloid₁₋₄₂ preparations that were either directly reconstituted in Me₂SO or pre-treated with HFIP prior to reconstitution. Activated caspase-3 was detected using a rabbit polyclonal antibody from Cell Signaling (Danvers, Mass.).

FIGS. 7A-B illustrate the effects of TGase on cell viability. FIG. 7A shows that retinoic acid (RA)-differentiated SH-SY5Y cells were treated with either 2.5, 5, or 10 μM β-amyloid₁₋₄₂ (BA) for 48 hours in the presence or absence of 25 μM monodansyl cadaverine (MDC). Cell viability was measured by the reduction of 3-(4,5-dimethylthiazole-2-yl)-2,5-diphenyltetrazolium bromide (MTT). There was a significant difference (p=0.022) among the groups treated with 10 μM BA, as well as between the groups treated with 5 μM BA (p=0.027). Inset depicts TGase expression in SH-SY5Y cells after 4-6 days of treatment with 20 μM RA. FIG. 7B shows that retinoic acid-differentiated cells were treated with H₂O₂ (20 μM) for 24 hours in the presence or absence of MDC and cell viability was assessed for the different conditions.

FIGS. 8A-B illustrate the effects of TGase on cellular apoptosis. FIG. 8A shows that SH-SH5Y cells grown in low serum were transiently transfected with either control vector, wild-type TGase, or the transamidation-defective TGase (C277V) mutant, and then exposed to 5 μM β-amyloid₁₋₄₂ (BA) for 48 hours. Bar graphs represent the percentage of apoptotic cells in control and BA-treated cells, and depict a significant increase in the death of BA-treated cells that have been transfected with wild-type TGase (p=0.016). Experimental conditions in FIG. 8B were similar to those described in FIG. 8A, except cells were treated with H₂O₂ (10 μM), rather than BA, for 24 hours.

FIGS. 9A-B show changes in transglutaminase activity during β-amyloid-induced apoptosis. FIG. 9A shows that treatment of RA-differentiated SH-SY5Y cells with 10 μM β-amyloid₁₋₄₂ (BA) over 48 hours resulted in the activation of caspase-3 (bottom panel), together with a significant increase in the levels of TGase in the particulate fraction (middle panel), as well as transamidation activity (top panel), as assayed by the cross-linking of 5-(biotin-amido)-pentylamine to proteins (see Example 5). Experimental conditions in FIG. 9B were similar to those described in FIG. 9A, except cells were treated with H₂O₂ rather than BA for 24 hours.

FIGS. 10A-D depict immunofluorescence and cell fractionation of TGase in control cells and cells treated with β-amyloid. Immunofluorescence staining for TGase in permeabilized SH-SY5Y cells treated with RA, using procedures similar to those previously described (Erickson et al., J. Biol. Chem. 271:26850-26854 (1996), which is hereby incorporated by reference in its entirety) (FIG. 10A), or in non-permeabilized cells treated with RA in the absence (FIG. 10B) and presence (FIG. 10C) of 10 μM β-amyloid₁₋₄₂ (BA 630 X). Treatment with β-amyloid₁₋₄₂ was for 48 hours at 37° C. Sucrose gradient cell fractionation (FIG. 10D; see Example 4) shows that TGase is predominantly present in the cytosolic fractions from SH-SY5Y cells (containing 0.25 to 0.9 M sucrose); however, when cells were incubated with β-amyloid₁₋₄₂ (BA), TGase appeared in the pelleted fraction together with aggregated BA.

FIGS. 11A-B illustrate examination of the cross-linking of β-amyloid by TGase. FIG. 11A shows that in-vitro transamidation reactions were performed for 60 minutes using 0.1 μg of guinea pig liver TGase (Sigma, St. Louis, Mo.) and 1 μg of β-amyloid₁₋₄₂ (BA), in the presence and absence of CaCl₂ and MDC, as described in Example 5. The immunoblot for BA shows rapid oligomerization due to TGase-catalyzed transamidation, which is stimulated by the addition of 500 μM Ca²⁺ and can be completely eliminated with the transamidation inhibitor MDC. FIG. 11B shows that β-amyloid (BA) is found in the particulate fractions from SH-SY5Y cells, together with TGase, but there are no detectable higher oligomers of BA due to TGase-catalyzed cross-linking. The band seen at the top of the gel does not arise from TGase-stimulated cross-linking of BA, as it is not affected when transamidation activity is inhibited by MDC.

### DETAILED DESCRIPTION OF THE INVENTION

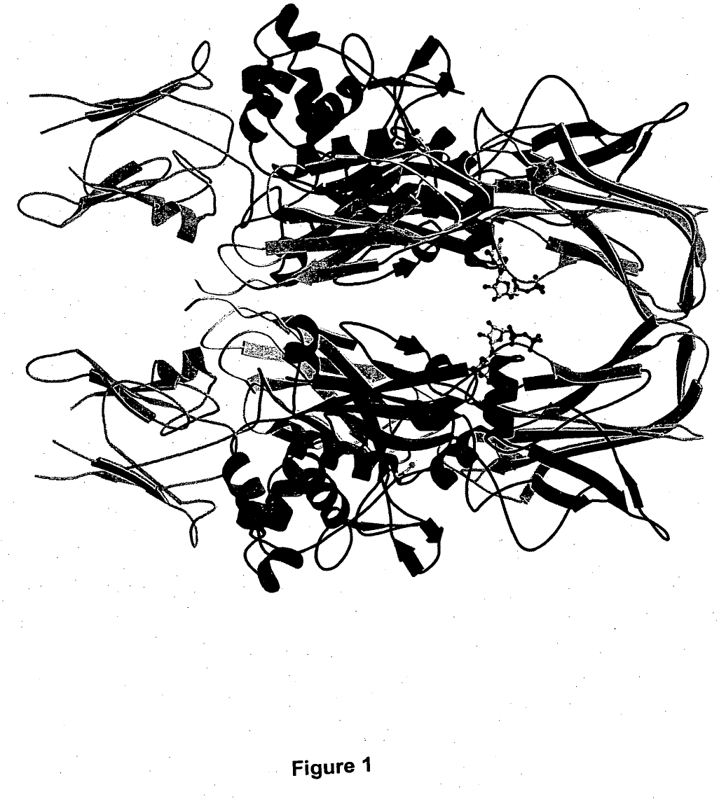

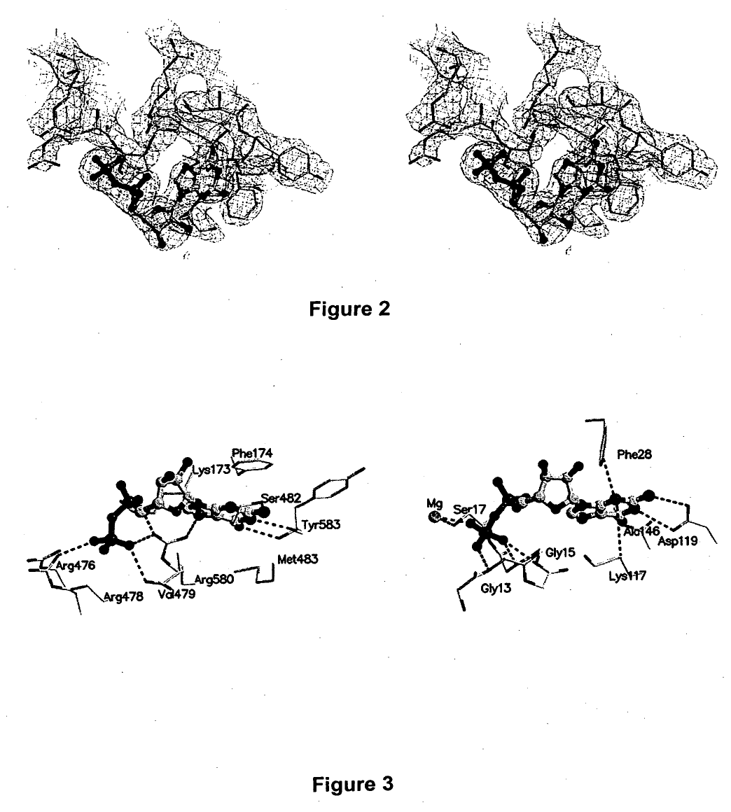

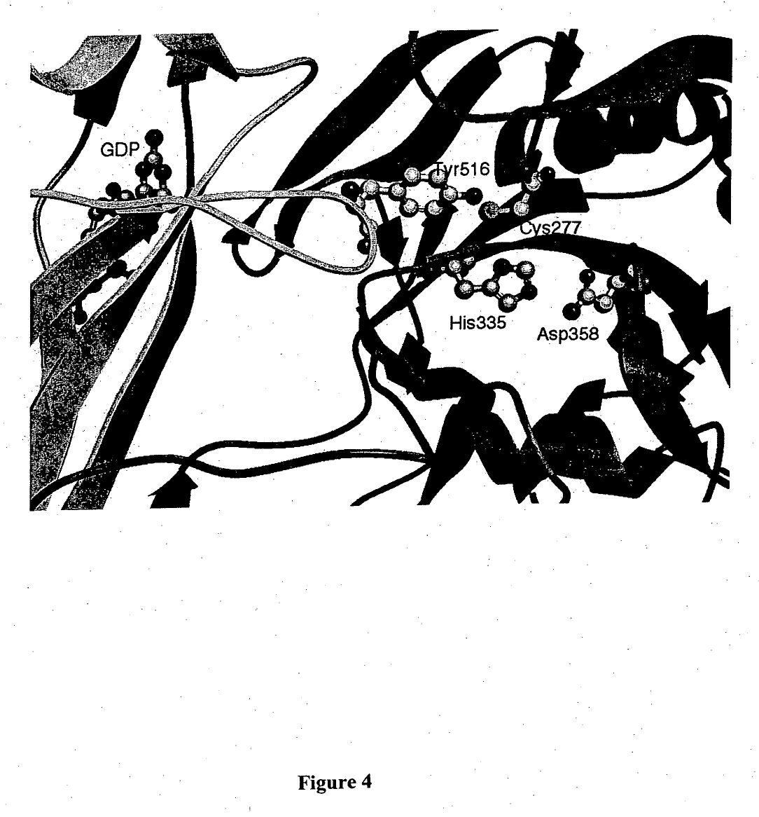

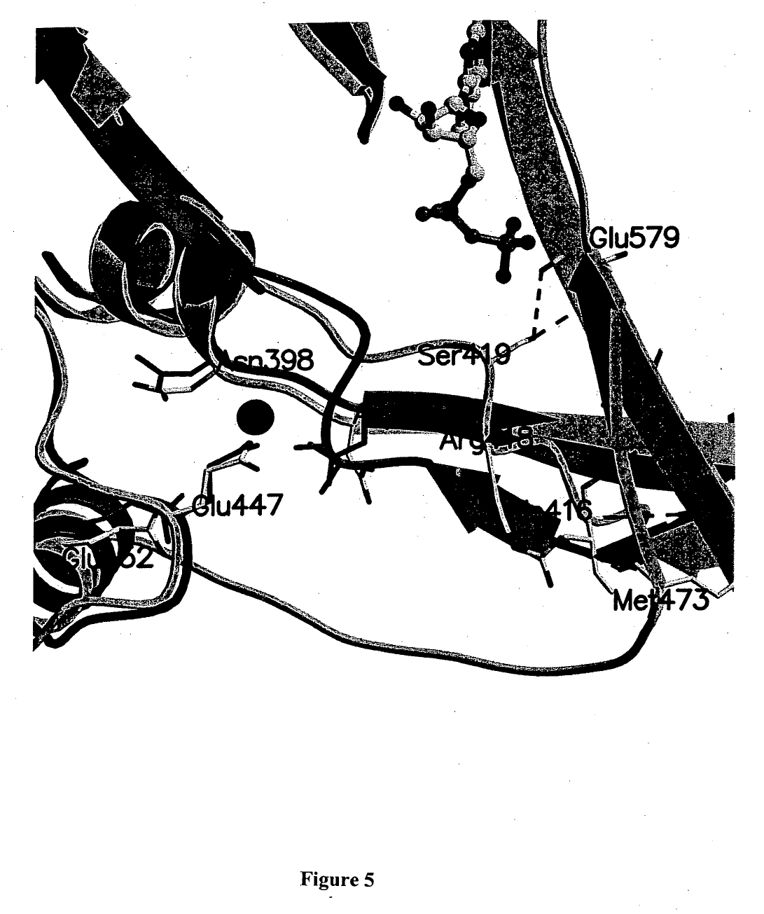

TGase is a Ca²⁺-dependent acyltransferase with roles in cellular differentiation, apoptosis, and other biological functions. In addition to being a transamidase, TGase undergoes a GTP-binding/GTPase cycle even though it lacks any obvious sequence similarity with canonical GTP-binding (G) proteins. Guanine nucleotide binding and Ca²⁺ concentration reciprocally regulate TGase's transamidation activity, with nucleotide binding being the negative regulator. FIGS. 1, 2, 3, 5, and 6 of U.S. Patent Application Publication No. US 2004/0259176 to Liu et al., which is hereby incorporated by reference in its entirety, illustrate the three-dimensional structure of human TGase complexed with GDP determined to 2.8-Å resolution by x-ray crystallography. (FIGS. 1, 2, 3, 5, and 6 of U.S. Patent Application Publication No. US 2004/0259176 to Liu et al. have been reproduced in the present application in black and white as FIGS. 1, 2, 3, 4, and 5, respectively.) Although the transamidation active site is similar to those of other known transglutaminases, the guanine nucleotide-binding site of TGase differs markedly from other G proteins. The structure of TGase suggests a structural basis for the negative regulation of transamidation activity by bound nucleotide, and the positive regulation of transamidation by Ca²⁺.

The present invention relates to a method of inhibiting beta-amyloid-induced death of neuronal cells in a subject. The method involves inhibiting human tissue transglutaminase in the subject under conditions effective to inhibit beta-amyloid-induced death of neuronal cells. In one embodiment of the present invention, the human tissue transglutaminase has the sequence according to SEQ ID NO: 1 as follows:

```
Met Ala Glu Glu Leu Val Leu Glu Arg Cys Asp Leu Glu Leu Glu Thr
    1 5 10 15
Asn Gly Arg Asp His His Thr Ala Asp Leu Cys Arg Glu Lys Leu Val
                          20 25 30
Val Arg Arg Gly Gln Pro Phe Trp Leu Thr Leu His Phe Glu Gly Arg
                  35 40 45
Asn Tyr Glu Ala Ser Val Asp Ser Leu Thr Phe Ser Val Val Thr Gly
          50 55 60
Pro Ala Pro Ser Gln Glu Ala Gly Thr Lys Ala Arg Phe Pro Leu Arg
  65 70 75 80
Asp Ala Val Glu Glu Gly Asp Trp Thr Ala Thr Val Val Asp Gln Gln
                                  85 90 95
Asp Cys Thr Leu Ser Leu Gln Leu Thr Thr Pro Ala Asn Ala Pro Ile
                        100 105 110
Gly Leu Tyr Arg Leu Ser Leu Glu Ala Ser Thr Gly Tyr Gln Gly Ser
                115 120 125
Ser Phe Val Leu Gly His Phe Ile Leu Leu Phe Asn Ala Trp Cys Pro
        130 135 140
Ala Asp Ala Val Tyr Leu Asp Ser Glu Glu Glu Arg Gln Glu Tyr Val
145 150 155 160
Leu Thr Gln Gln Gly Phe Ile Tyr Gln Gly Ser Ala Lys Phe Ile Lys
                                165 170 175
Asn Ile Pro Trp Asn Phe Gly Gln Phe Glu Asp Gly Ile Leu Asp Ile
                        180 185 190
Cys Leu Ile Leu Leu Asp Val Asn Pro Lys Phe Leu Lys Asn Ala Gly
                195 200 205
Arg Asp Cys Ser Arg Arg Ser Ser Pro Val Tyr Val Gly Arg Val Val
        210 215 220
Ser Gly Met Val Asn Cys Asn Asp Asp Gln Gly Val Leu Leu Gly Arg
225 230 235 240
Trp Asp Asn Asn Tyr Gly Asp Gly Val Ser Pro Met Ser Trp Ile Gly
                                245 250 255
Ser Val Asp Ile Leu Arg Arg Trp Lys Asn His Gly Cys Gln Arg Val
                        260 265 270
Lys Tyr Gly Gln Cys Trp Val Phe Ala Ala Val Ala Cys Thr Val Leu
                275 280 285
Arg Cys Leu Gly Ile Pro Thr Arg Val Val Thr Asn Tyr Asn Ser Ala
        290 295 300
His Asp Gln Asn Ser Asn Leu Leu Ile Glu Tyr Phe Arg Asn Glu Phe
305 310 315 320
Gly Glu Ile Gln Gly Asp Lys Ser Glu Met Ile Trp Asn Phe His Cys
                                325 330 335
Trp Val Glu Ser Trp Met Thr Arg Pro Asp Leu Gln Pro Gly Tyr Glu
                        340 345 350
Gly Trp Gln Ala Leu Asp Pro Thr Pro Gln Glu Lys Ser Glu Gly Thr
                355 360 365
Tyr Cys Cys Gly Pro Val Pro Val Arg Ala Ile Lys Glu Gly Asp Leu
        370 375 380
Ser Thr Lys Tyr Asp Ala Pro Phe Val Phe Ala Glu Val Asn Ala Asp
385 390 395 400
Val Val Asp Trp Ile Gln Gln Asp Asp Gly Ser Val His Lys Ser Ile
                                405 410 415
Asn Arg Ser Leu Ile Val Gly Leu Lys Ile Ser Thr Lys Ser Val Gly
                        420 425 430
Arg Asp Glu Arg Glu Asp Ile Thr His Thr Tyr Lys Tyr Pro Glu Gly
                435 440 445
Ser Ser Glu Glu Arg Glu Ala Phe Thr Arg Ala Asn His Leu Asn Lys
        450 455 460
Leu Ala Glu Lys Glu Glu Thr Gly Met Ala Met Arg Ile Arg Val Gly
465 470 475 480
Gln Ser Met Asn Met Gly Ser Asp Phe Asp Val Phe Ala His Ile Thr
                                485 490 495
Asn Asn Thr Ala Glu Glu Tyr Val Cys Arg Leu Leu Leu Cys Ala Arg
                        500 505 510
Thr Val Ser Tyr Asn Gly Ile Leu Gly Pro Glu Cys Gly Thr Lys Tyr
                515 520 525
Leu Leu Asn Leu Asn Leu Glu Pro Phe Ser Glu Lys Ser Val Pro Leu
        530 535 540
Cys Ile Leu Tyr Glu Lys Tyr Arg Asp Cys Leu Thr Glu Ser Asn Leu
545 550 555 560
Ile Lys Val Arg Ala Leu Leu Val Glu Pro Val Ile Asn Ser Tyr Leu
                                565 570 575
Leu Ala Glu Arg Asp Leu Tyr Leu Glu Asn Pro Glu Ile Lys Ile Arg
                        580 585 590
Ile Leu Gly Glu Pro Lys Gln Lys Arg Lys Leu Val Ala Glu Val Ser
                595 600 605
Leu Gln Asn Pro Leu Pro Val Ala Leu Glu Gly Cys Thr Phe Thr Val
        610 615 620
Glu Gly Ala Gly Leu Thr Glu Glu Gln Lys Thr Val Glu Ile Pro Asp
625 630 635 640
Pro Val Glu Ala Gly Glu Glu Val Lys Val Arg Met Asp Leu Leu Pro
                                645 650 655
Leu His Met Gly Leu His Lys Leu Val Val Asn Phe Glu Ser Asp Lys
                        660 665 670
Leu Lys Ala Val Lys Gly Phe Arg Asn Val Ile Ile Gly Pro Ala
                675 680 685
```

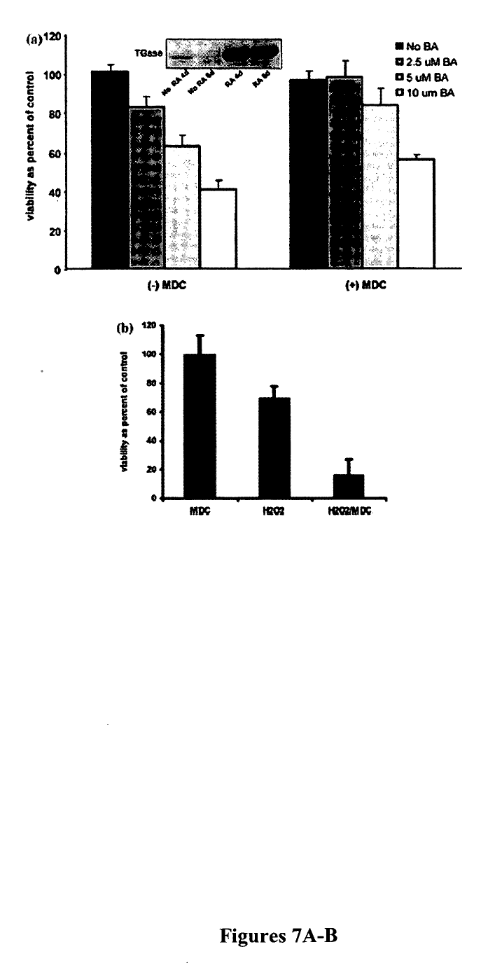

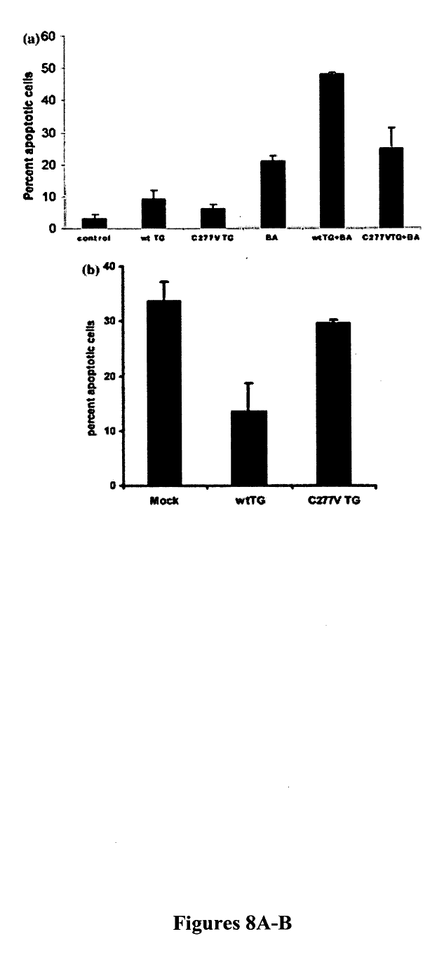

The inhibition can be achieved with a compound which binds to one or more molecular surfaces of the human tissue transglutaminase having a three dimensional crystal structure defined by the structural coordinates set forth in FIG. 7 of U.S. Patent Application Publication No. US 2004/0259176, which is hereby incorporated by reference in its entirety.

In one embodiment of the present invention, the molecular surfaces of the human tissue transglutaminase include atoms surrounding one or more of residues Lys-173, Phe-174, Arg-476, Arg-478, Val-479, Ser-482, Met-483, Arg-580, Leu-582, or Tyr-583 of SEQ ID NO: 1.

The inhibition of tissue transglutaminase can be carried out by administering an inhibitor of tissue transglutaminase orally, intradermally, intramuscularly, intraperitoneally, intravenously, subcutaneously, or intranasally. The inhibitor compounds of the present invention may be administered alone or with suitable pharmaceutical carriers, and can be in solid or liquid form, such as tablets, capsules, powders, solutions, suspensions, or emulsions.

The inhibitor compounds may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The inhibitor compounds may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Another aspect of the present invention relates to a method for identifying candidate compounds suitable for inhibiting beta-amyloid-induced death of neuronal cells in a subject. The method involves identifying compounds which are capable of binding to human tissue transglutaminase as candidate compounds suitable for inhibiting beta-amyloid-induced death of neuronal cells in a subject. In another embodiment, the method further involves contacting human tissue transglutaminase with a compound, prior to the step of identifying.

The present invention also relates to a method for designing a compound suitable for inhibiting beta-amyloid-induced death of neuronal cells in a subject. The method first involves providing a three-dimensional structure of a crystallized human tissue transglutaminase. Then, a compound having a three-dimensional structure which will bind to one or more molecular surfaces of the human tissue transglutaminase is designed. The three dimensional structure of the crystallized tissue transglutaminase may be defined by the structural coordinates set forth in FIG. 7 of U.S. Patent Application Publication No. US 2004/0259176, which is hereby incorporated by reference in its entirety. In inhibiting beta-amyloid-induced cell death of neuronal cells, the compounds designed by this method or pharmaceutical compositions containing such compounds (as well as a pharmaceutical carrier) are dosed and administered by the modes described above.

### EXAMPLES

The following examples are provided to illustrate embodiments of the present invention but are by no means intended to limit its scope.

### Example 1

### Cell Differentiation and Effects of Treatment with β-Amyloid and H₂O₂

To induce differentiation, human SH-SY5Y cells were cultured in phenol red-free, low serum media with 20 μM all-trans-retinoic acid (RA; Sigma) every other day for 6 days. When assaying cell viability, the cells were plated in 96-well dishes at a seeding density of approximately 5×10³ cells per well, differentiated with 20 μM RA, and then treated with either media alone, 25 μM monodansyl cadaverine (MDC), β-amyloid₁₋₄₂ (2.5, 5, and 10 μM) (HPLC-purified from American Peptide, Sunnyvale, Calif.), or with 25 μM MDC and different concentrations of β-amyloid₁₋₄₂. Typically, β-amyloid₁₋₄₂ was reconstituted in 2.5 mM Me₂SO and then further diluted into cell culture media, using conditions that have been reported to give rise primarily to β-amyloid oligomers and large aggregates rather than fibrils (Dahlgren et al., J. Biol. Chem. 277:32046-32053 (2002); Stine et al., J. Biol. Chem. 278:11612-11622 (2003), which are hereby incorporated by reference in their entirety). However, in some cases, β-amyloid₁₋₄₂ was first dissolved in 100% hexafluoroisopropanolol to ensure the elimination of any preexisting aggregates ((Dahlgren et al., J. Biol. Chem. 277:32046-32053 (2002); Stine et al., J. Biol. Chem. 278:11612-11622 (2003), which are hereby incorporated by reference in their entirety) and then this solvent was removed by evaporation prior to reconstituting the peptide in Me₂SO. Cells were incubated with the reconstituted β-amyloid₁₋₄₂ for 48 hours and cell viability was then assayed by the reduction of 3-(4,5-dimethylthiazole-2-yl)-2,5-diphenyltetrazolium bromide (MTT), as previously described in Miller et al., Brain Res. 963:43-56 (2003), which is hereby incorporated by reference in its entirety. The effects of H₂O₂ (10-30 μM) on cell viability were assayed in a similar manner (Tamagno et al., Exp Neurol 180:144-155 (2003), which is hereby incorporated by reference in its entirety). Values for untreated controls were set at 100% viability and each treatment was assessed as a percentage of the control values (+/−SD). Each treatment was performed in triplicate and evaluated by using a one-way analysis of variation (ANOVA) with Turkey's post hoc analysis to determine differences between the groups with the a value set at 0.05.

### Example 2

### Construction of Wild-Type and Mutant TGase Expression Vectors and Transfections

Construction of the pcDNA3 Myc-tagged vector used herein has been described in Tu et al., J. Biol. Chem. 278:49293-49300 (2003), which is hereby incorporated by reference in its entirety. Insertion of the TGase cDNA into the vector was accomplished by performing BamHI and EcoRI restriction-site digestion of the previously generated pTRE TGase (wild-type) vector (Antonyak et al., J. Biol. Chem. 276:33582-33587 (2001), which is hereby incorporated by reference in its entirety) to excise the cDNA encoding wild-type TGase, followed by subcloning it into the pcDNA3 Myc-tagged vector using T4 DNA ligase (Invitrogen, Carlsbad, Calif.). The pcDNA3-Myc-TGase vector was used to generate the transamidation-defective (C277V) mutant with the Quikchange site-directed mutagenesis kit (Stratagene, La Jolla, Calif.). Each TGase construct was sequenced to confirm the presence or absence of the mutation. The TGase constructs were transfected, using Lipofectamine (Invitrogen), into SH-SY5Y cells that had been cultured and exposed to low serum conditions for 4 days. Three hour transfections were performed, and then the cells were placed in low serum media for 4 hours, after which they were treated with 5 μM β-amyloid₁₋₄₂ for 48 hours or remained in low serum media as controls.

### Example 3

### Immunofluorescence and Nuclear Condensation or Blebbing Assays

Cells were plated in 6-well dishes with poly-lysine-treated glass coverslips. The cells were differentiated for 6 days with 20 μM RA. After differentiation, treatment consisted of either media alone, 25 μM MDC, 10 μM β-amyloid₁₋₄₂, or both 25 μM MDC and 10 μM β-amyloid₁₋₄₂ for 48 hours. The cells were then fixed with 3.7% formaldehyde in phosphate-buffered saline (PBS), pH 7.4, for 20 minutes. The cells were washed briefly with PBS, permeabilized with 0.1% Triton-X 100 in PBS for 10 minutes, and washed again. PBS-containing BSA was added to each well for 2 hours as a blocking reagent and the cells were then incubated with polyclonal rabbit-TGase antibody (Neomarkers, Fremont, Calif.) for 2 hours at room temperature. The wells were washed (three times) for 5 minutes with PBS and then secondary antibody was added, i.e. anti-rabbit Oregon Green (Molecular Probes, Carlsbad, Calif.). After 60 minutes of incubation with the secondary antibody, the nuclei were stained using Hoescht 3222 that was added from the Vybrant apoptosis kit according to the manufacturer's instructions (Molecular Probes). The cells were washed (three times) in PBS, mounted on slides, and visualized with a Zeiss microscope and ZeissVision software (Zeiss, Thornwood, N.Y.).

Immunofluorescence was also performed 48 hours after transient transfections with TGase constructs. Cells were fixed as described in the preceding paragraph and immunofluorescence was performed on cells expressing Myc-tagged wild-type TGase or the TGase (C277V) mutant. In these experiments, the primary antibody used was anti-mouse Myc antibody (Sigma) and the secondary antibody was anti-mouse Oregon Green (Molecular Probes). Cells expressing the various TGase constructs were counted from three separate experiments (over 100 cells from each slide were scored) after β-amyloid₁₋₄₂ treatment and the apoptotic rate was assessed relative to the percentage of apoptosis for untreated (transfected) cells. Cells were stained with Hoescht 3222 and those cells exhibiting either nuclear condensation and/or blebbing were designated as apoptotic. Statistical analysis was performed comparing the three groups using a single way ANOVA and Tukey's post hoc analysis, with an α value set at 0.05.

### Example 4

### Cell Fractionation

SH-SY5Y cells, treated with and without β-amyloid₁₋₄₂ for 48 hours, were lysed by freeze/thawing using liquid nitrogen in a 0.25 M sucrose solution containing 1 mM dithiothreitol, 1 μg/ml aprotinin, 1 μg/ml leupeptin and 100 μM phenylmethylsulfonyl fluoride. The cell lysates were then layered on top of a sucrose gradient (0.4-1.5 M sucrose) and centrifuged for 3 hours at 39K. Fractions (100 μl) were collected and then mixed with 100 μl of 20% SDS and 50 μL of 5×loading buffer. The samples were then subjected to SDS-PAGE and Western blotting using anti-TGase (Neomarkers) and anti-β-amyloid (6E10, Signet Laboratories, Inc., Dedham, Mass.) antibodies.

### Example 5

### Transglutaminase Activity Assays

The in vivo transamidation assays were performed as previously described in Antonyak et al., J. Biol. Chem. 279:41461-41467 (2004), which is hereby incorporated by reference in its entirety. Briefly, RA-treated SH-SY5Y cells were exposed to various concentrations of β-amyloid₁₋₄₂ (2.5-10 μM) for 48 hours, or H₂O₂ (10-30 μM) for 24 hours, and then were incubated with and without 1 mM 5-(biotin-amido)-pentylamine (BPA) for 16 hours. The cells were lysed and the soluble and particulate fractions (˜50 μg total protein) were subjected to SDS-PAGE, transferred to PVDF membranes, and the membranes were blocked overnight at 4° C. in BBST (100 mM boric acid, 20 mM sodium borate, 0.01% SDS, 0.01% Tween-20, and 80 mM NaCl) containing 3% BSA. The membranes were then incubated with horseradish-peroxidase-conjugated streptavidin (2 mg/ml; Pierce Inc., Rockford, Ill.) (diluted 1:2000) in BBST for 2 hours at room temperature, followed by washing (three times) for 20 minutes with BBST. The protein-5-(biotin-amido)-pentylamine conjugates were then visualized on radiograph film after exposing the membranes to chemiluminescence reagent (ECL, Amersham Corp., Louisville, Colo.).

The in vitro transamidation reactions were performed with one or all of the following: 1 μg of β-amyloid₁₋₄₂, 0.1 μg of guinea pig liver TGase (Sigma), 500 μM CaCl₂, and/or 50 μM MDC. Each reaction was performed in 50 mM Tris-HCl, pH 7.2, and incubated for 60 minutes at 37° C. The reactions were stopped by the addition of an equal volume of 20% SDS and 10 μl of Laemmeli's sample buffer and immediately boiled for 20 minutes to break up any non-covalent aggregation of β-amyloidi₁₋₄₂. SDS-PAGE was performed on the samples, followed by blotting onto nitrocellulose membranes (Amersham). The membranes were immunoblotted with anti-β-amyloid antibody.

### Example 6

### Examination of the Effects of β-Amyloid₁₋₄₂ on a Differentiated Neuronal Cell Line

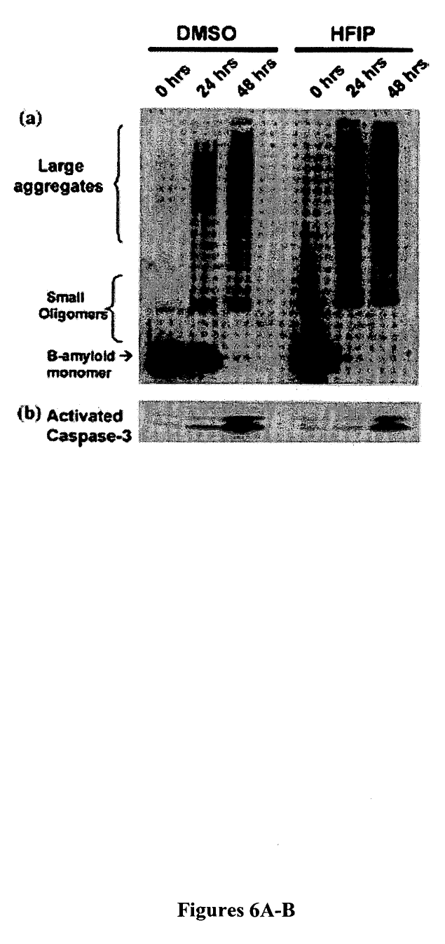

To examine the functional interplay between TGase and β-amyloid in a model cell system, the human SH-SY5Y cell line was chosen, since these cells undergo neuronal differentiation and up-regulate TGase expression in response to RA treatment. Given that it has been reported that the method of treatment of β-amyloid₁₋₄₂ may influence its cellular activity (Dahlgren et al., J. Biol. Chem. 277:32046-32053 (2002); Stine et al., J. Biol. Chem. 278:11612-11622 (2003), which are hereby incorporated by reference in their entirety), different approaches for reconstituting this peptide were first assessed. One approach involved reconstituting the β-amyloid₁₋₄₂ peptide directly into Me₂SO, prior to its dilution into cell culture media and addition to the SH-SY5Y cells (see Example 1 above). A second approach was to first treat β-amyloid₁₋₄₂ with hexafluoroisopropanol (HFIP), prior to reconstituting it in Me₂SO, as this has been suggested to remove any preexisting higher oligomeric structures in the β-amyloid₁₋₄₂ stocks that might contribute to experimental variability (Dahlgren et al., J. Biol. Chem. 277:32046-32053 (2002); Stine et al., J. Biol. Chem. 278:11612-11622 (2003), which are hereby incorporated by reference in their entirety). Both reconstitution approaches yielded similar outcomes, with the primarily monomeric β-amyloid₁₋₄₂ being converted to larger oligomeric forms after 48 hours of incubation at room temperature (FIG. 6A), which induced an apoptotic response as read-out by caspase-3 activation (FIG. 6B). Thus, in the experiments with SH-SY5Y cells described below, a stock solution of 2.5 M β-amyloid₁₋₄₂ that was directly reconstituted in Me₂SO and then incubated for 48 hours was used to ensure the formation of the oligomeric species.

SH-SY5Y cells cultured in low serum-media normally exhibited very little expression of TGase. Upon addition of the differentiation factor RA, a significant up-regulation of TGase was observed with a maximal response occurring after 4 days of treatment (FIG. 7A, inset). Incubation of the SH-SY5Y cells with β-amyloid₁₋₄₂ for 48 hours had no effect on TGase expression (either in the absence or presence of RA). However, treatment of RA-differentiated SH-SY5Y cells with β-amyloid₁₋₄₂ caused a dose-dependent cytotoxic response (FIG. 7A). When the effects of β-amyloid₁₋₄₂ were examined in the presence of MDC, a competitive (substrate) inhibitor of TGase-catalyzed transamidation, there was a consistent reduction in cell death, suggesting that the transamidation activity of TGase contributed to the β-amyloid-induced cytotoxic response.

It has been proposed that the neurotoxic effects caused by β-amyloid were due to its ability to elicit oxidative stress through the generation of H₂O₂ (Tamagno et al., Free Radic Biol Med 35:45-58 (2003); Tamagno et al., Exp Neurol 180:144-155 (2003), which are hereby incorporated by reference in their entirety). Thus, it was also examined whether blocking the transamidation activity of TGase reduced the extent of cytotoxicity that occurred when treating SH-SY5Y cells with this reactive oxygen intermediate. Interestingly, it was found that this was not the case. FIG. 7B shows that, upon treatment of RA-differentiated SH-SY5Y cells with H₂O₂, cell viability was reduced by 30-40%. However, addition of the TGase inhibitor MDC did not protect against the H₂O₂-induced effects, but rather greatly enhanced cytotoxicity. These findings were similar to other results showing that TGase often confers protection against cytotoxic and apoptotic insults and that inhibition of TGase by MDC promotes cell death (Antonyak et al., J. Biol. Chem. 276:33582-33587 (2001); Boehm et al., J. Biol. Chem. 277:20127-20130 (2002); Antonyak et al., J. Biol. Chem. 279:41461-41467 (2004), which are hereby incorporated by reference in their entirety). Indeed, these and other results described below, demonstrate that TGase activity does not serve as a general contributor to cell death, but instead acts specifically to augment the neurotoxic effects of β-amyloid.

### Example 7

### Effects of Expressing Wild-Type and Transamidation-Defective TGase on β-amyloid₁₋₄₂-Induced Apoptosis

To specifically establish that TGase plays a role in β-amyloid-induced apoptosis, SH-SY5Y cells were incubated in low serum-media for 6 days (in the absence of RA treatment), followed by transient transfection with either empty vector, or vectors encoding Myc-tagged wild-type TGeese, or the transamidation-deficient TGase (C277V) mutant, before being exposed to β-amyloid₁₋₄₂. Control cells showed little tendency to undergo apoptosis in the absence or presence of TGase expression (FIG. 8A). As expected, incubation with 5 μM β-amyloid₁₋₄₂ increased the percentage of cells undergoing apoptosis, compared to control cells. However, the apoptotic effects obtained with β-amyloid₄₂ were markedly enhanced in cells expressing wild-type TGase, whereas no significant change in the levels of β-amyloid-induced apoptosis were observed in cells expressing the transamidation-defective TGase mutant.

Again, opposite results were found when examining the effects of TGase on H₂O₂-induced apoptosis (FIG. 8B). Expression of wild-type TGase provided significant protection against H₂O₂-induced apoptosis, whereas the transamidation-defective mutant was essentially ineffective. Overall, these findings were consistent with those obtained from MDC-treated cells, suggesting that the transamidation activity of TGase specifically augmented the ability of β-amyloid₁₋₄₂ to induce cell death in SH-SY5Y cells.

### Example 8

### Treatment with β-Amyloid but not H₂O₂ Markedly Enhances the Transamidation Activity Detected in the Membrane/Particulate Fractions of Cells

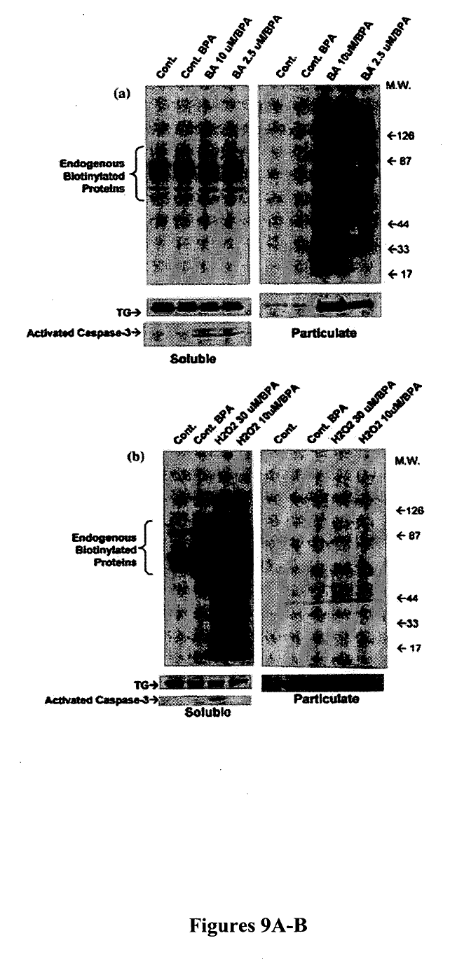

Under conditions where β-amyloid caused SH-SY5Y cells to undergo apoptosis, there was an accompanying increase in the transamidation activity in the membrane/particulate fractions. As shown in FIG. 9A, upon treating cells with 2.5-10 μM β-amyloid₁₋₄₂, which led to caspase activation (bottom panel) and apoptosis, the levels of TGase in the membrane/particulate fractions were dramatically increased (middle panel). This was accompanied by a marked enhancement in the production of biotin-amido-pentylamine (BPA) conjugates (top panel) in the membrane/particulate fractions, indicative of increased transamidation activity. These results clearly differed from what occurred in cells exposed to H₂O₂ (FIG. 9B). Treatment of the cells with the reactive oxygen intermediate also resulted in caspase activation (i.e., at 30 μM H₂O₂) (bottom panel), but under these conditions there was a significant increase in cytosolic transamidation activity (top panel), with little detectable TGase (middle panel) or transamidation activity in the particulate fractions.

### Example 9

### Further Examination of the Effects of β-Amyloid on the Cellular Localization of TGase

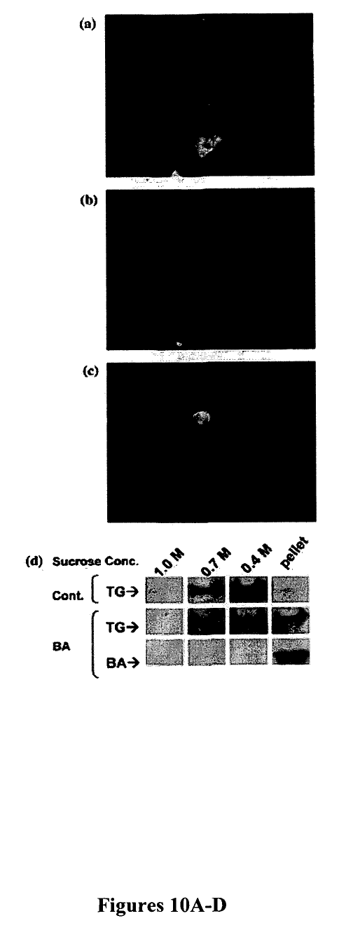

Immunofluorescence experiments were then performed to determine how β-amyloid influences the cellular localization of TGase. In RA-treated cells that were permeabilized, TGase was distributed throughout the cytoplasm (FIG. 10A), whereas in non-permeabilized cells, TGase staining was significantly diminished (FIG. 10B). Upon incubating the cells with β-amyloid₁₋₄₂, TGase was clearly detected along the surface of the non-permeabilized cells (FIG. 10C), indicating that β-amyloid induced the movement of TGase to the plasma membrane, such that it was accessible to antibody staining from outside of the cells.

Sucrose density sedimentation experiments also showed that in control cells which were not treated with β-amyloid, TGase was predominantly present in the cytosolic fractions, sedimenting between 0.4-0.9 M sucrose (some examples of gradient fractions containing 0.4, 0.7, and 1 M sucrose are shown in FIG. 10D). However, in cells that were incubated with β-amyloid₁₋₄₂, a significant amount of TGase was present in the pelleted (particulate) material together with aggregates of β-amyloid (see FIG. 10D, pellet). Thus, taken together, the results presented in FIGS. 9 and 10 suggest that β-amyloid specifically recruits TGase to the cell surface.

### Example 10

### Is β-Amyloid a Transamidation Substrate for TGase in Cells Undergoing Apoptosis?

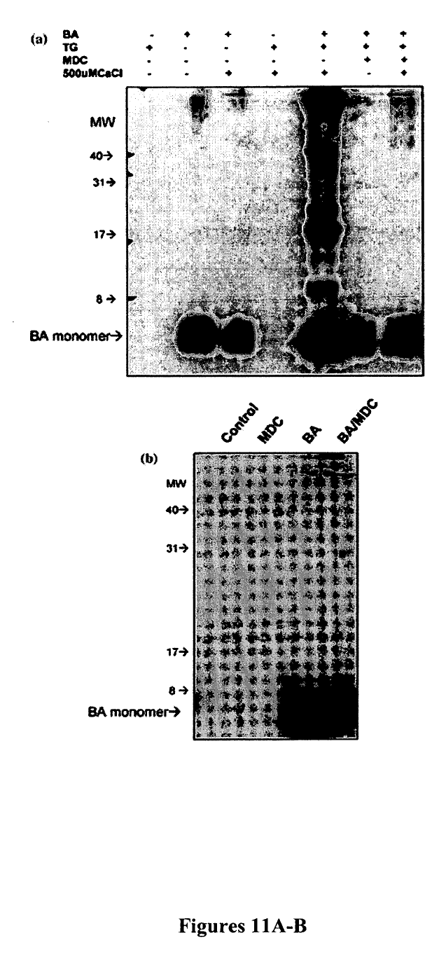

How might β-amyloid-induced changes in the cellular localization of TGase contribute to a stronger apoptotic response? One possibility was that TGase might use β-amyloid as a transamidation substrate (Johnson et al., Brain Res. 751:323-329 (1997), which is hereby incorporated by reference in its entirety), and in doing so, enhance its apoptotic activity. Indeed, β-amyloid₁₋₄₂ is an effective transamidation substrate for TGase in vitro (FIG. 11A); also, see Dudek et al., Brain Res. 651:129-133 (1994), which is hereby incorporated by reference in its entirety), as significant transamidation of β-amyloid₁₋₄₂ can be detected at Ca²⁺ levels below 1 mM. However, thus far, evidence of TGase-catalyzed oligomerization of β-amyloid₁₋₄₂ (i.e., due to the cross-linking of β-amyloid) has not been detected either in the cell culture media or in the particulate fractions of SH-SY5Y cells treated with β-amyloid₁₋₄₂ and RA (FIG. 11B). Therefore, rather than TGase-catalyzed transamidation of β-amyloid being responsible for the augmentation of β-amyloid-induced apoptosis in neuronal cells, a distinct and still to be identified transamidation substrate(s) is likely to be involved.

### Example 11

### Effects of TGase on β-Amyloid₁₋₄₂-Induced Apoptosis

TGase has been implicated in a number of cellular processes and disease states, but exactly how TGase functions in these different biological contexts is still being elucidated. A particularly interesting question has concerned the role of this GTP-binding protein/acyl transferase in cell survival versus programmed cell death, and how it fits into neurodegenerative diseases. Antonyak et al. have suggested that TGase contributes to cell survival, both through its GTP-binding and transamidation activities (Antonyak et al., J. Biol. Chem. 276:33582-33587 (2001), which is hereby incorporated by reference in its entirety), as well as through some type of interplay with the retinoblastoma (Rb) protein (Boehm et al., J. Biol. Chem. 277:20127-20130 (2002), which is hereby incorporated by reference in its entirety). However, others have proposed that TGase directly participates in programmed cell death (Piacentini et al., Int. J. Cancer 52:271-278 (1992); Oliverio et al., Mol. Cell Biol. 17:6040-6048 (1997); Melino et al., FEBS Lett. 430:59-63 (1998), which are hereby incorporated by reference in their entirety). Along these lines, there also has been a good deal of circumstantial evidence suggesting that TGase may play a role in neurodegenerative disorders, particularly Alzheimer's and Huntingtons' diseases, through the aberrant crosslinking of β-amyloid and other proteins linked to these pathological conditions (Appelt et al., J. Histochem. Cytochem. 44:1421-1427 (1996); Johnson et al., Brain Res. 751:323-329 (1997); Zhang et al., Acta Neuropathol. (Berl) 96:395-400 (1998); Maggio et al., Brain Res. Bull. 56:173-182 (2001); Nemes et al., Neurobiol. Aging 22:403-406 (2001); Bonelli et al., Neurobiol. Dis. 11:106-110 (2002), which are hereby incorporated by reference in their entirety). The present application describes studies further examining the relationship between TGase and β-amyloid, in an effort to determine whether TGase contributes to or antagonizes β-amyloid's neurotoxic effects.

Interestingly, it was found that while β-amyloid₁₋₄₂ did not directly stimulate TGase expression or activity in the SH-SY5Y neuronal cell line, it caused a change in its cellular localization, resulting in TGase being detected in the plasma membrane fractions in close proximity to β-amyloid, as well as in the pelleted material together with aggregated β-amyloid₁₋₄₂ during cell fractionation. Moreover, TGase enhanced the neurotoxic responses triggered by β-amyloid₁₋₄₂, as treatment with the transamidation-competitive inhibitor, MDC, partially reversed β-amyloid-induced cytotoxicity. In addition, the ectopic expression of wild-type TGase in SH-SY5Y cells significantly increased the abilities of sub-optimal doses of β-amyloid₁₋₄₂ to cause apoptosis, whereas expression of a transamidation-defective TGase mutant showed no effect. It is especially important to note that these effects by TGase were specific for β-amyloid-treatment and were not observed when cytotoxicity and apoptosis were induced by H₂O₂, a reactive oxygen intermediate that has been shown to be neurotoxic and to help mediate the apoptotic effects of β-amyloid (Tamagno et al., Free Radic Biol Med 35:45-58 (2003); Tamagno et al., Exp Neurol 180:144-155 (2003), which are hereby incorporated by reference in their entirety). In fact, TGase conferred protection against H₂O₂-induced cytotoxicity and apoptosis, similar to what had been earlier observed when cells were challenged with other apoptotic insults (Antonyak et al., J. Biol. Chem. 276:33582-33587 (2001); Boehm et al., J. Biol. Chem. 277:20127-20130 (2002), which are hereby incorporated by reference in their entirety).

The results disclosed in the present application also indicated that TGase was not simply functioning directly downstream from β-amyloid in a signaling pathway leading to cell death, as MDC did not block the ability of β-amyloid₁₋₄₂ to activate caspases nor to stimulate c-Jun kinase (JNK) activity. Moreover, expression of the transamidation-defective TGase mutant did not function as a dominant-negative inhibitor of β-amyloid-induced apoptosis, as would have been expected if TGase were acting downstream from β-amyloid in a common signaling pathway. Thus, these findings indicate that while TGase expression and/or activity is often up-regulated as a protective measure against cellular stress, in the specific case of β-amyloid, the (β-amyloid-induced) recruitment of TGase to the cell surface results in deleterious effects on cell viability.

It has been proposed that the cellular location of TGase is linked to its roles in apoptosis versus survival, whereby TGase's localization to the nucleus was suggested to confer a survival benefit (Lesort et al., J. Biol. Chem. 273:11991-11994 (1998); Milakovic et al., J. Biol. Chem. 279:8715-8722 (2004), which are hereby incorporated by reference in their entirety). However, the nuclear levels of TGase have not been found to be altered in response to β-amyloid₁₋₄₂. This raises the question of how β-amyloid-induced changes in the localization of TGase lead to enhanced cell death? It can be speculated that TGase might use β-amyloid as a transamidation substrate, such that the ensuing modification of β-amyloid enhanced its neurotoxic activity. There, in fact, have been reports that β-amyloid is susceptible to TGase-catalyzed transamidation (Dudek et al., Brain Res. 651:129-133 (1994), which is hereby incorporated by reference in its entirety), and β-amyloid₁₋₄₂ is a very effective transamidation substrate in vitro. However, the cross-linking of β-amyloid₁₋₄₂ in the particulate fractions containing TGase has not been detected from β-amyloid-treated neuronal cells, although these fractions exhibited significantly enhanced transamidation activity. This leads one to suspect that the β-amyloid-directed recruitment of TGase, perhaps to sites at or near the cell surface, enables it to catalyze the transamidation of another protein whose cross-linking is detrimental to cell viability and survival.

Therefore, a rather interesting picture has emerged regarding the actions of TGase. Namely, the up-regulation of TGase in neuronal cells, which under normal conditions of cellular differentiation as well as in response to some cellular insults (e.g. H₂O₂), serves a beneficial function to ensure cell viability, in fact becomes deleterious to survival when cells are exposed to β-amyloid₁₋₄₂. These findings now raise a number of intriguing questions. For example, what are the mechanics by which β-amyloid₁₋₄₂ “recruits” TGase to the cell surface, what is the identity of the putative transamidation substrate(s) whose modification significantly contributes to apoptosis, and what is the mechanism by which some cellular insults like H₂O₂ cause an activation of TGase's transamidation activity in the cytosol and how does this confer protection against neurotoxicity?

Although the invention has been described in detail, for the purpose of illustration, it is understood that such detail is for that purpose and variations can be made therein by those skilled in the art without departing from the spirit and scope of the invention which is defined by the following claims.

What is claimed:

1. A method of inhibiting beta-amyloid-induced death of neuronal cells in a subject, said method comprising:
inhibiting human tissue transglutaminase in the subject under conditions effective to inhibit beta-amyloid-induced death of neuronal cells.

2. The method according to claim 1, wherein the human tissue transglutaminase has the sequence according to SEQ ID NO: 1.

3. The method according to claim 2, wherein said inhibiting is achieved with a compound which binds to one or more molecular surfaces of the human tissue transglutaminase having a three-dimensional crystal structure defined by the structural coordinates set forth in FIG. 7 of U.S. Patent Application Publication No. US 2004/0259176, which is hereby incorporated by reference in its entirety.

4. The method according to claim 3, wherein the molecular surfaces of the human tissue transglutaminase comprise atoms surrounding one or more of residues Lys-173, Phe-174, Arg-476, Arg-478, Val-479, Ser-482, Met-483, Arg-580, Leu-582, or Tyr-583 of SEQ ID NO: 1.

5. The method according to claim 1, wherein said inhibiting is carried out by administering an inhibitor of human tissue transglutaminase orally, intradermally, intramuscularly, intraperitoneally, intravenously, subcutaneously, or intranasally.

6. A method for identifying candidate compounds suitable for inhibiting beta-amyloid-induced death of neuronal cells in a subject, said method comprising:
identifying compounds which are capable of binding to human tissue transglutaminase as candidate compounds suitable for inhibiting beta-amyloid-induced death of neuronal cells in a subject.

7. The method according to claim 6, further comprising:
contacting human tissue transglutaminase with a compound, prior to said identifying.

8. The method according to claim 6, wherein the human tissue transglutaminase has the sequence according to SEQ ID NO: 1.

9. The method according to claim 8, wherein the compound is capable of binding to one or more molecular surfaces of the human tissue transglutaminase having a three-dimensional crystal structure defined by the structural coordinates set forth in FIG. 7 of U.S. Patent Application Publication No. US 2004/0259176, which is hereby incorporated by reference in its entirety.

10. The method according to claim 9, wherein the molecular surfaces of the human tissue transglutaminase comprise atoms surrounding one or more of residues Lys-173, Phe-174, Arg-476, Arg-478, Val-479, Ser-482, Met-483, Arg-580, Leu-582, or Tyr-583 of SEQ ID NO: 1.

11. A method for designing a compound suitable for inhibiting beta-amyloid-induced death of neuronal cells in a subject, said method comprising:
providing a three-dimensional structure of a crystallized human tissue transglutaminase; and
designing a compound having a three-dimensional structure which will bind to one or more molecular surfaces of the human tissue transglutaminase.

12. The method according to claim 11, wherein the human tissue transglutaminase has the sequence according to SEQ ID NO: 1.

13. The method according to claim 12, wherein the three-dimensional structure of a crystallized human tissue transglutaminase is defined by the structural coordinates set forth in FIG. 7 of U.S. Patent Application Publication No. US 2004/0259176, which is hereby incorporated by reference in its entirety.

14. The method according to claim 13, wherein the molecular surfaces of the human tissue transglutaminase comprise atoms surrounding one or more of residues Lys-173, Phe-174, Arg-476, Arg-478, Val-479, Ser-482, Met-483, Arg-580, Leu-582, or Tyr-583 of SEQ ID NO: 1.

15. A compound designed by the method of claim 11.

16. A pharmaceutical composition comprising the compound of claim 15 and a pharmaceutical carrier.

17. A compound suitable for inhibiting beta-amyloid-induced death of neuronal cells in a subject, said compound having a three-dimensional structure which will bind to one or more molecular surfaces of human tissue transglutaminase having a three-dimensional crystal structure defined by the structural coordinates set forth in FIG. 7 of U.S. Patent Application Publication No. US 2004/0259176, which is hereby incorporated by reference in its entirety.

18. The compound according to claim 17, wherein the molecular surfaces of the tissue transglutaminase comprise atoms surrounding one or more of residues Lys-173, Phe-174, Arg-476, Arg-478, Val-479, Ser-482, Met-483, Arg-580, Leu-582, or Tyr-583 of SEQ ID NO: 1.


SEQUENCE LISTING (1 sequence)

SEQ ID NO: 1 - protein, 687 aa, Homo sapiens
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA